(19) United States
(12) Patent Application Publication
(10) Pub. No.: US 2021/0212607 A1
(43) Pub. Date: Jul. 15, 2021

(54) Component Concentration Measuring Device

(71) Applicant: Nippon Telegraph and Telephone Corporation, Tokyo (JP)

(72) Inventors: Yujiro Tanaka, Tokyo (JP); Masahito Nakamura, Tokyo (JP); Daichi Matsunaga, Tokyo (JP); Michiko Seyama, Tokyo (JP)

(21) Appl. No.: 17/051,604

(22) PCT Filed: Apr. 19, 2019
(86) PCT No.: PCT/JP2019/016808

§ 371 Date: Oct. 29, 2020

(30) Foreign Application Priority Data
May 1, 2018 (JP) 2018-088064

(51) Int. Cl. A61B 5/145; A61B 5/00

(52) U.S. Cl. CPC A61B 5/14532; A61B 5/6826; A61B 5/6816; A61B 5/0095

(57) ABSTRACT

A light source unit emits beam light of a wavelength that is absorbed by glucose. A light application control unit gives multiple-application of the beam light emitted by the light source unit to a site of measurement. A detection unit detects each of a plurality of photoacoustic signals that are generated at the site of measurement due to the multiple-application of the beam light by the light application control unit. A processing unit averages the plurality of photoacoustic signals detected by the detection unit.

### CROSS-REFERENCE TO RELATED APPLICATIONS

This application is a national phase entry of PCT Application No. PCT/JP2019/016808, filed on Apr. 19, 2019, which claims priority to Japanese Application No. 2018-088064, filed on May 1, 2018, which applications are hereby incorporated herein by reference.

### TECHNICAL FIELD

The present invention relates to a component concentration measurement device for non-invasively measuring glucose concentration.

### BACKGROUND

In terms of determining a dose of insulin for a diabetes patient or preventing diabetes, it is important to know (measure) blood sugar level. The blood sugar level is the concentration of glucose in blood, and as a way of measuring this kind of component concentration, a photoacoustic method is well known (see Patent Literature 1).

When a certain amount of light (an electromagnetic wave) is applied to a living body, the applied light is absorbed by molecules contained in the living body. As a result, target molecules for measurement in a portion applied with the light are locally heated to expand and generate a sound wave. The pressure of the sound wave depends on the amount of molecules that absorb the light. The photoacoustic method is an approach that measures this sound wave to measure the amount of molecules in the living body. A sound wave is a pressure wave that propagates within a living body and has a property of being resistant to scattering compared to an electromagnetic wave; the photoacoustic method can be regarded as a suitable way for measuring blood components in a living body.

Measurement by the photoacoustic method enables continuous monitoring of the glucose concentration in blood. In addition, measurement with the photoacoustic method does not require blood sample and causes no discomfort in a subject of measurement.

### CITATION LIST

### Patent Literature

Patent Literature 1: Japanese Patent Laid-Open No. 2010-104858

### SUMMARY

### Technical Problem

A site on a human body that is subjected to this type of measurement can change in thickness over time. For example, the thickness of skin and the like can locally change before and after eating or drinking. When the thickness or the like of the site of measurement thus changes, however, a measurement result of glucose measurement within human body by the photoacoustic method will change. As the measurement result changes due to such a change in human body, it can happen that the concentrations are actually the same when results that were measured at different times are different or that the concentrations are actually different when results that were measured at different times are the same, which hinders an accurate measurement.

In order to solve these drawbacks, an object of embodiments of the present invention is to suppress decrease in measurement accuracy that is caused by a change in human body over time in measurement of glucose in human body by the photoacoustic method.

### Means for Solving the Problem

A component concentration measurement device according to embodiments of the present invention includes: a light source unit that emits beam light of a wavelength that is absorbed by glucose; a light application control unit that gives multiple-application of the beam light to a site of measurement; a detection unit that detects each of a plurality of photoacoustic signals that are generated at the site of measurement due to the multiple-application of the beam light by the light application control unit; and a processing unit that averages the plurality of photoacoustic signals detected by the detection unit.

In the component concentration measurement device, the light application control unit may give multiple-application of the beam light by applying the beam light at a plurality of individually different locations in the site of measurement.

In the component concentration measurement device, the light application control unit may apply the beam light at a plurality of individually different locations in the site of measurement by scanning the beam light emitted by light source unit.

In the component concentration measurement device, the light application control unit may give multiple-application of the beam light by applying the beam light at individually different times.

In the component concentration measurement device, the detection unit may detect each of the plurality of photoacoustic signals individually, and the processing unit may determine an average of the photoacoustic signals individually detected by the detection unit.

In the component concentration measurement device, the light application control unit may apply the beam light at a plurality of locations in the site of measurement within a detection region of the detection unit, and the processing unit may be the detection unit, and the detection unit may also average the plurality of photoacoustic signals by detecting all of the plurality of photoacoustic signals in the detection region.

### Effects of Embodiments of the Invention

As described above, in accordance with embodiments of the present invention, each of multiple photoacoustic signals that are generated at the site of measurement due to the multiple-application of beam light by the light application control unit is detected by the detection unit, and the detected multiple photoacoustic signals are averaged by the processing unit. Thus, it provides an advantageous effect of suppressing decrease in the measurement accuracy that is caused by a change in human body over time in measurement of glucose in human body by the photoacoustic method.

### BRIEF DESCRIPTION OF THE DRAWINGS

FIG. 1 is configuration diagram showing a configuration of a component concentration measurement device in Embodiment 1 of the present invention.

FIG. 2 is a plan view for describing a scanning state of beam light 121.

FIG. 3 is configuration diagram showing more detailed configurations of a light source unit 101 and a detection unit 103 in an embodiment of the present invention.

FIG. 4 is configuration diagram showing a configuration of a component concentration measurement device in Embodiment 2 of the present invention.

FIG. 5 is configuration diagram showing a configuration of a component concentration measurement device in Embodiment 3 of the present invention.

### DETAILED DESCRIPTION OF ILLUSTRATIVE EMBODIMENTS

Component concentration measurement devices according to embodiments of the present invention are described below.

### Embodiment 1

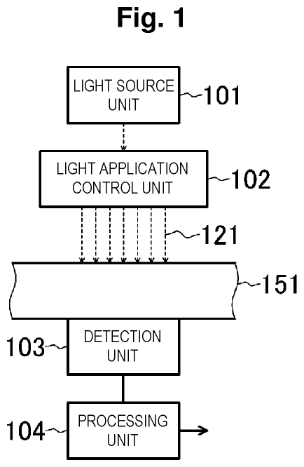

First, referring to FIG. 1, a component concentration measurement device in Embodiment 1 of the present invention is described. The component concentration measurement device includes a light source unit 101, a light application control unit 102, a detection unit 103, and a processing unit 104.

The light source unit 101 emits beam light of a wavelength that is absorbed by glucose. The light application control unit 102 gives multiple-application of the beam light emitted by the light source unit 101 to a site of measurement 151. The site of measurement 151 is a portion of a human body, like a finger or an ear lobe, for example. In Embodiment 1, the light application control unit 102 gives multiple-application of the beam light by applying the beam light emitted by the light source unit 101 at multiple individually different locations in the site of measurement 151.

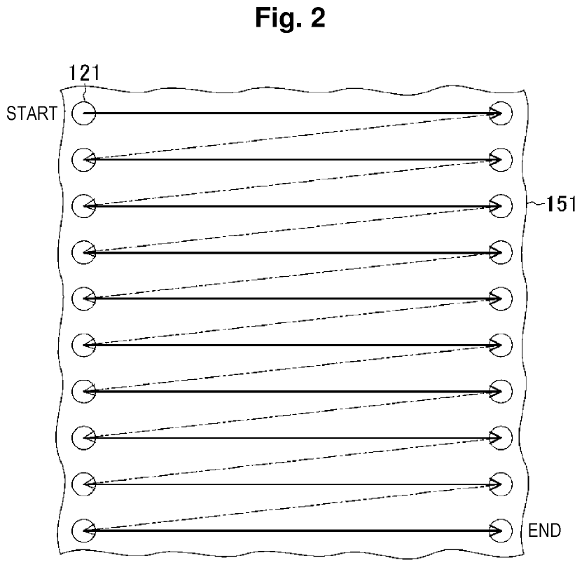

For example, as shown in FIG. 2, the light application control unit 102 scans (raster-scans) the beam light emitted the light source unit 101, thereby applying beam light 121 at multiple individually different locations in the site of measurement 151. For example, a beam diameter of the beam light 121 is about 100 μm. For example, in a square region of about 3 mm per side, the light application control unit 102 scans the beam light 121 and applies it at multiple individually different locations in the site of measurement 151. The light application control unit 102 may carry out this scanning with a galvano mirror, for example. The light application control unit 102 may also carry out the scanning of the beam light 121 by means of a well-known MEMS mirror, for example.

The light application control unit 102 also splits an incident light beam into multiple light beams via an optical fiber array or the like and applies them to individually different locations in the site of measurement 151.

The detection unit 103 detects each of multiple photoacoustic signals that are generated at the site of measurement 151 due to the multiple-application of beam light by the light application control unit 102. The processing unit 104 averages the multiple photoacoustic signals detected by the detection unit 103. For example, the detection unit 103 detects each of the multiple photoacoustic signals individually, and the processing unit 104 determines and outputs an average of the multiple photoacoustic signals individually detected by the detection unit 103. For example, the detection unit 103 detects each of the multiple photoacoustic signals individually by moving to the locations where the beam light is applied. Alternatively, each of the multiple photoacoustic signals may be individually detected by disposing multiple detection units 103 in a region where the beam light is applied.

In measurement of glucose in human body with the photoacoustic method, the state of the site of measurement 151 at different times changes due to effect of body temperature, ambient temperature, amount of moisture at the site of measurement 151, blood flow at the site of measurement 151, and the like. Such a change in the state of the site of measurement 151 leads to a lower accuracy of measurement results. As opposed to this, Embodiment 1 enables suppression of decrease in measurement accuracy even if the state of the site of measurement 151 changes with lapse of time. This is attributed to the processing unit 104 averaging the multiple photoacoustic signals that were measured at different locations in a predefined region in the site of measurement 151.

The processing unit 104 may also compute an average without using a maximum measured value and a minimum measured value so that a variance of multiple measurement results falls in a predetermined range. It is also possible to preliminarily perform measurements in multiple regions and then perform a measurement in a region where the variance of the multiple measurement results obtained in the regions falls in a predetermined range.

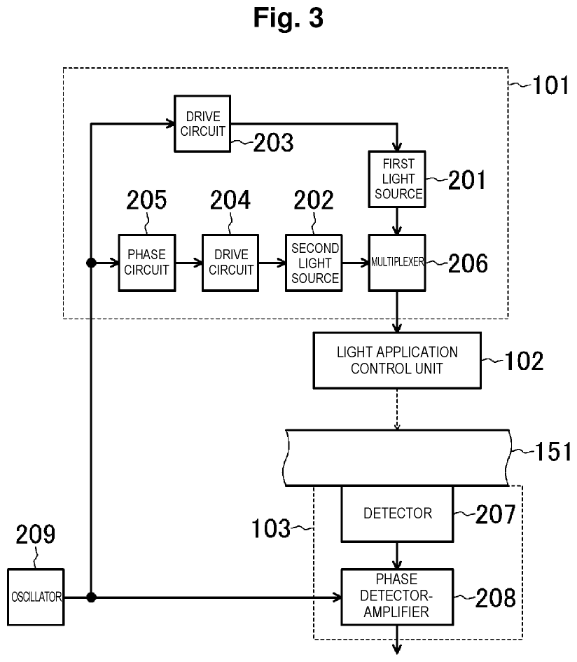

Now referring to FIG. 3, the light source unit 101 and the detection unit 103 are described in more detail. The light source unit 101 includes a first light source 201, a second light source 202, a drive circuit 203, a drive circuit 204, a phase circuit 205, and a multiplexer 206. The detection unit 103 includes a detector 207, a phase detector-amplifier 208, and an oscillator 209.

The oscillator 209 is connected to each of the drive circuit 203, the phase circuit 205, and the phase detector-amplifier 208 via signal wires. The oscillator 209 sends signals to each of the drive circuit 203, the phase circuit 205, and the phase detector-amplifier 208.

The drive circuit 203 receives the signal sent from the oscillator 209, and supplies driving electric power to the first light source 201, which is connected by a signal wire, to cause the first light source 201 to emit light. The first light source 201 is a semiconductor laser, for example.

The phase circuit 205 receives the signal sent from the oscillator 209, and sends a signal generated by giving a phase shift of 180° to the received signal to the drive circuit 204, which is connected by a signal wire.

The drive circuit 204 receives the signal sent from the phase circuit 205, and supplies driving electric power to the second light source 202, which is connected by a signal wire, to cause the second light source 202 to emit light. The second light source 202 is a semiconductor laser, for example.

The first light source 201 and the second light source 202 output light of different wavelengths from each other and direct their respective output light to the multiplexer 206 via light wave transmission means. For the first light source 201 and the second light source 202, the wavelength of light of one of them is set to a wavelength that is absorbed by glucose, while the wavelength of light of the other is set to a wavelength that is absorbed by water. Their respective wavelengths are also set such that degrees of their absorption will be equivalent.

The light output by the first light source 201 and the light output by the second light source 202 are multiplexed in the multiplexer 206 and are incident onto the light application control unit 102 as one light beam. Upon incidence of the light beam, the light application control unit 102 scans the incident light beam, for example, to apply it to the site of measurement 151. Upon incidence of the light beam, the light application control unit 102 also splits the incident light beam into multiple light beams, for example, and applies them at individually different locations in the site of measurement 151. In the site of measurement 151 with the multiple light beams thus applied at individually different locations, a photoacoustic signal is generated in the inside of each location applied with the light beam.

The detector 207 individually detects the each of photoacoustic signals generated in the site of measurement 151, converts them into electric signals, and sends them to the phase detector-amplifier 208, which is connected by a signal wire. The phase detector-amplifier 208 receives a synchronization signal necessary for synchronous detection sent from the oscillator 209, and also receives the electric signals proportional to the multiple photoacoustic signals being sent from the detector 207. The phase detector-amplifier 208 performs synchronous detection, amplification, and filtering on each of the received electric signals, and outputs each electric signal proportional to each photoacoustic signal individually.

The first light source 201 outputs light that has been intensity-modulated in synchronization with an oscillation frequency of the oscillator 209. In contrast, the second light source 202 outputs light that has been intensity-modulated with the oscillation frequency of the oscillator 209 and in synchronization with the signal that has gone through a phase shift of 180° in the phase circuit 205.

The intensity of the signal output by the phase detector-amplifier 208 is proportional to the amounts of components (glucose, water) in the site of measurement 151. This is because the light that is output by each of the first light source 201 and the second light source 202 is proportional to the amount of light that was absorbed by the components in the site of measurement 151. From a measured value of the strength of the signal thus output, a component concentration derivation unit (not shown) determines the amount of the target component (glucose) in blood at the site of measurement 151.

As mentioned above, the light output by the first light source 201 and the light output by the second light source 202 have been intensity-modulated with signals of the same frequency. Consequently, there is no effect of unevenness in frequency characteristics of a measurement system, which is problematic in the case of intensity modulation with signals of multiple frequencies.

Meanwhile, non-linear dependence on absorption coefficient that exits in measured values of photoacoustic signals, which is problematic in measurement by the photoacoustic method, can be solved by performing measurement using light of multiple wavelengths that gives an equal absorption coefficient as described above (see Patent Literature 1).

### Embodiment 2

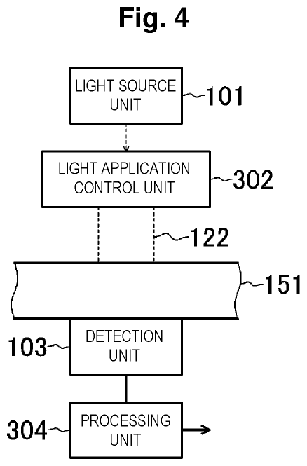

Next, referring to FIG. 4, a component concentration measurement device in Embodiment 2 of the present invention is described. The component concentration measurement device includes a light source unit 101, a light application control unit 302, a detection unit 103, and a processing unit 304.

The light source unit 101 emits beam light of a wavelength that is absorbed by glucose. The light application control unit 302 gives multiple-application of the beam light 122 to a site of measurement 151. In Embodiment 2, the light application control unit 302 gives multiple-application of the beam light 122 by applying the beam light 122 at individually different times. The site of measurement 151 is a portion of a human body, like a finger or an ear lobe, for example. The beam diameter of the beam light 122 is of a size enough to apply light to substantially the entirety of a region detectable by the detection unit 103 (a square region of about 3 mm per side), for example. It is also possible to create a light application state that is substantially the same as a state in which the beam light 122 of such a large beam diameter as mentioned above is applied in the following manner: the light application control unit 302 may scan and apply the incident light beam at high speed in a square region of about 3 mm per side, for example. In this case, a scanning speed for completing one scan may be an amount of time such that a distance for which a photoacoustic signal (sound wave) travels within the site of measurement 151 is equal to 1/10 wavelength or smaller.

The detection unit 103 detects each of multiple photoacoustic signals that are generated at different times in the site of measurement 151 as a result of the multiple-application of the beam light at different times by the light application control unit 302. The processing unit 34 averages the multiple photoacoustic signals that have been detected by the detection unit 103 respectively at different times. The processing unit 304 determines and outputs an average of the multiple photoacoustic signals that have been detected by the detection unit 103 respectively at different times.

In measurement of glucose in human body with the photoacoustic method, the state of the site of measurement 151 at different times changes due to effect of body temperature, ambient temperature, amount of moisture at the site of measurement 151, blood flow at the site of measurement 151, and the like. Such a change in the state of the site of measurement 151 leads to a lower accuracy of measurement results. As opposed to this, Embodiment 2 enables suppression of decrease in the measurement accuracy even if the state of the site of measurement 151 changes with lapse of time. This is attributed to the averaging of multiple photoacoustic signals that are measured at different times in a predefined region in the site of measurement 151.

### Embodiment 3

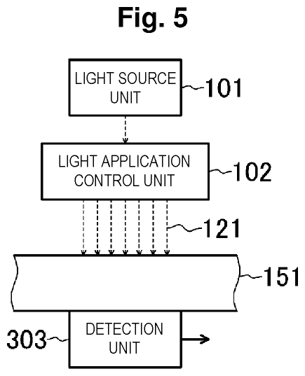

Next, referring to FIG. 5, a component concentration measurement device in Embodiment 3 of the present invention is described. The component concentration measurement device includes a light source unit 101, a light application control unit 102, and a detection unit 303.

The light source unit 101 emits beam light of a wavelength that is absorbed by glucose. The light application control unit 102 gives multiple-application of the beam light to a site of measurement 151. These arrangements are similar to the Embodiment 1 described earlier. In Embodiment 3, the light application control unit 102 applies the beam light 121 at multiple locations in the site of measurement 151 so that they correspond to a square detection region of the detection unit 303 that is about 3 mm per side, for example. The detection unit 303 then simultaneously detects all of the multiple photoacoustic signals that are generated in response to the multiple beam light 121 being applied in a predetermined region. The multiple photoacoustic signals detected in the detection region of the detection unit 303 are converted to electric signals by the detection unit 303 after being averaged and are output. In Embodiment 3, functionality of the processing unit in Embodiment 1 is implemented with the detection unit 303.

In Embodiment 3 as well, decrease in measurement accuracy can be suppressed even if the state of the site of measurement 151 changes with lapse of time as in Embodiment 1. This is attributed to averaging the multiple photoacoustic signals measured at different locations in a predetermined region in the site of measurement 151.

As described above, in accordance with embodiments of the present invention, each of multiple photoacoustic signals that are generated at the site of measurement due to the multiple-application of beam light by the light application control unit is detected by the detection unit, and the detected multiple photoacoustic signals are averaged by the processing unit. It can suppress decrease in the measurement accuracy that is caused by a change in human body over time in measurement of glucose in human body by the photoacoustic method.

It will be apparent that the present invention is not limited to the above-described embodiments but many variations and combinations may be made by ordinarily skilled persons in the art within the technical idea of the invention.

### REFERENCE SIGNS LIST

101 light source unit

102 light application control unit

103 detection unit

104 processing unit

151 site of measurement.

1.-6. (canceled)

7. A component concentration measurement device comprising:
a light source that emits beam light having a wavelength that is absorbed by glucose;
a light application controller that controls multiple applications of the beam light to a site of measurement;
a detector that detects a plurality of photoacoustic signals that are generated at the site of measurement due to the multiple applications of the beam light by the light application controller; and
a processor that averages the plurality of photoacoustic signals detected by the detector.

8. The component concentration measurement device according to claim 7, wherein the light application controller controls the multiple applications of the beam light by causing the beam light to be applied at a plurality of individually different locations on the site of measurement.

9. The component concentration measurement device according to claim 8, wherein:
the plurality of individually different locations on the site of measurement is within a detection region of the detector;
the processor and the detector are a same device; and
the detector averages the plurality of photoacoustic signals by detecting each of the plurality of photoacoustic signals in the detection region.

10. The component concentration measurement device according to claim 7, wherein the light application controller causes the beam light to be applied at a plurality of different locations on the site of measurement by scanning the beam light emitted by light source.

11. The component concentration measurement device according to claim 7, wherein the light application controller controls the multiple applications of the beam light by causing the beam light to be applied at different times.

12. The component concentration measurement device according to claim 7, wherein:
the detector detects each of the plurality of photoacoustic signals individually; and
the processor determines an average of the plurality of photoacoustic signals individually detected by the detector.

13. A method comprising:
emitting beam light having a wavelength that is absorbed by glucose;
applying multiple applications of the beam light to a site of measurement;
detecting a plurality of photoacoustic signals that are generated at the site of measurement due to the multiple applications of the beam light; and
averaging the plurality of photoacoustic signals.

14. The method according to claim 13, wherein applying the multiple applications of the beam light comprises applying the beam light at a plurality of individually different locations on the site of measurement.

15. The method according to claim 14, wherein:
the plurality of individually different locations on the site of measurement is within a detection region of a detector; and
averaging the plurality of photoacoustic signals and detecting the plurality of photoacoustic signals comprises using the detector.

16. The method according to claim 13, wherein applying the multiple applications of the beam light comprises applying the beam light at a plurality of different locations on the site of measurement by scanning the beam light.

17. The method according to claim 13, wherein applying the multiple applications of the beam light comprises applying the beam light at different times.

18. The method according to claim 13, wherein detecting the plurality of photoacoustic signals comprises detecting each of the plurality of photoacoustic signals individually.
